(19) United States
(12) Patent Application Publication
(10) Pub. No.: US 2012/0225946 A1
(43) Pub. Date: Sep. 6, 2012

(54) CHOLINE FENOFIBRATE DELAYED RELEASE COMPOSITIONS

(76) Inventor: Bernard Charles Sherman, Toronto (CA)

(21) Appl. No.: 13/395,141

(22) PCT Filed: Sep. 9, 2010
(86) PCT No.: PCT/CA10/01392

§ 371 Date: May 23, 2012

Related U.S. Application Data
(60) Provisional application No. 61/240,885, filed on Sep. 9, 2009.

(51) Int. Cl. A61K 31/192; A61P 7/02

(52) U.S. Cl. 514/571

(57) ABSTRACT

Described are solid pharmaceutical compositions for oral administration comprising choline fenofibrate and an amount of an acid sufficient to substantially reduce a rate of dissolution of choline fenofibrate in an aqueous environment at pH 4.5 to less than about 50% in 2 hours. Processes for making the solid pharmaceutical composition for oral administration comprising: (a) mixing choline fenofibrate with an amount of an acid sufficient to substantially reduce a rate of dissolution of choline fenofibrate in an aqueous environment at pH 4.5 to less than about 50% in 2 hours; and (b) preparing a dosage form suitable for oral administration are also provided.

### TECHNICAL FIELD

This invention relates to the field of pharmaceutical formulations and in particular to pharmaceutical formulations comprising choline fenofibrate.

### BACKGROUND

Fenofibric acid and its salts are known to be useful for inhibition of platelet aggregation and reduction of triglyceride levels.

U.S. Pat. Nos. 4,179,515 and 4,235,895 disclose the preparation of fenofibric acid.

U.S. Pat. No. 4,372,954 discloses the moroxydine salt of fenofibric acid.

Spanish patent ES 474039 discloses the use of the cinnarizine salt; and the sodium salt has also been disclosed (Bosca et al., Photochemistry and Photobiology, 1999, 70(6), 853-857).

U.S. Pat. No. 7,259,186 discloses other salts of fenofibric acid, including specifically the choline, ethanolamine, diethanolamine, piperazine, calcium, and tromethamine salts.

Capsules made using choline fenofibrate are now sold on the U.S. market under the trade name Trilipix™, in strengths of 45 mg and 135 mg, expressed as fenofibric acid equivalent. Each capsule contains a multitude of small tablets of strength 11.25 mg, expressed as fenofibric acid, so that each 45 mg capsule contains 4 tablets, and each 135 mg capsule contains 12 tablets. The capsules are labeled as being delayed-release capsules. A delayed release dosage form is understood to be a dosage form which does not release a substantial portion of the active content for dissolution and absorption until it passes through the stomach and reaches the small intestine.

Gastric fluid is acidic, having a pH of up to about 4.5, and intestinal fluid is more basic, having pH of about 5.5 or higher. Choline fenofibrate is insoluble in acidic media having low pH, but solubility increases with pH. At pH of 4.5 and above, a tablet comprised substantially of choline fenofibrate will exhibit rapid dissolution. In order to prevent substantial dissolution in the stomach at pH 4.5, but permit dissolution when the tablets reach the small intestine, the tablets in Trilipix™ capsules comprise, in addition to choline fenofibrate, an enteric polymer. The enteric polymer is understood to mean a polymer which is insoluble in aqueous media at pH of 4.5 and lower, but soluble at higher pH. Such compositions comprising an enteric polymer are also disclosed in U.S. Pat. No. 7,259,186.

### SUMMARY

The present invention is based, at least in part, on the production of a delayed-release composition for oral administration made using choline fenofibrate that does not require the use of an enteric polymer.

The present invention is based, at least in part, on a sustained-release solid composition comprising choline fenofibrate without an enteric polymer. Such a composition may be made by using choline fenofibrate mixed with an acid that serves to reduce pH and thus inhibit dissolution in the stomach, at pH of up to about pH 4.5, while still enabling faster dissolution in the small intestine where pH is higher.

In illustrative embodiments of the present invention, there is provided a solid pharmaceutical composition for oral administration comprising choline fenofibrate and an amount of an acid sufficient to substantially reduce a rate of dissolution of choline fenofibrate in an aqueous environment at pH 4.5 to less than about 50% in 2 hours.

In illustrative embodiments of the present invention, there is provided the solid pharmaceutical composition described herein wherein the acid has a solubility in water of less than 2 parts per 50 parts of water at 20 degrees Celsius.

In illustrative embodiments of the present invention, there is provided the solid pharmaceutical composition described herein wherein the acid is fumaric acid.

In illustrative embodiments of the present invention, there is provided the solid pharmaceutical composition described herein wherein the aqueous environment is gastrointestinal fluid.

In illustrative embodiments of the present invention, there is provided the solid pharmaceutical composition described herein further comprising at least one additional pharmaceutically acceptable excipient.

In illustrative embodiments of the present invention, there is provided the solid pharmaceutical composition described herein wherein the at least one additional pharmaceutically acceptable excipient is selected from at least one of the group consisting of: binders, lubricants, flow agents and mixtures thereof.

In illustrative embodiments of the present invention, there is provided the solid pharmaceutical composition described herein wherein the at least one additional pharmaceutically acceptable excipient comprises microcrystalline cellulose.

In illustrative embodiments of the present invention, there is provided the solid pharmaceutical composition described herein wherein the at least one additional pharmaceutically acceptable excipient comprises magnesium stearate.

In illustrative embodiments of the present invention, there is provided the solid pharmaceutical composition described herein wherein the at least one additional pharmaceutically acceptable excipient comprises colloidal silicon dioxide.

In illustrative embodiments of the present invention, there is provided the solid pharmaceutical composition described herein wherein the pharmaceutical composition has a choline fenofibrate dissolution characteristic, when tested in USP apparatus 2, at 100 rpm at 37 degrees Celsius of not more than about 40% at 2 hours in 900 mL of 0.05M phosphate buffer, pH 4.5.

In illustrative embodiments of the present invention, there is provided the solid pharmaceutical composition described herein wherein the pharmaceutical composition has a choline fenofibrate dissolution characteristic, when tested in USP apparatus 2, at 100 rpm at 37 degrees Celsius of not less than about 50% at 2 hours in 900 mL of 0.05M phosphate buffer pH 6.8.

In illustrative embodiments of the present invention, there is provided a solid pharmaceutical composition for oral administration prepared by a process comprising: (a) mixing choline fenofibrate with an amount of an acid sufficient to substantially reduce a rate of dissolution of choline fenofibrate in an aqueous environment at pH 4.5 to less than about 50% in 2 hours; and (b) preparing a dosage form suitable for oral administration.

In illustrative embodiments of the present invention, there is provided the solid pharmaceutical composition for oral administration prepared by a process described herein wherein the acid has a solubility in water of less than 2 parts per 50 parts of water at 20 degrees Celsius.

In illustrative embodiments of the present invention, there is provided the solid pharmaceutical composition for oral administration prepared by a process described herein wherein the acid is fumaric acid.

In illustrative embodiments of the present invention, there is provided the solid pharmaceutical composition for oral administration prepared by a process described herein further comprising mixing additional pharmaceutically acceptable excipients prior to preparing the dosage form suitable for oral administration.

In illustrative embodiments of the present invention, there is provided the solid pharmaceutical composition for oral administration prepared by a process described herein wherein the at least one additional pharmaceutically acceptable excipient is selected from at least one of the group consisting of: binders, lubricants, flow agents and mixtures thereof.

In illustrative embodiments of the present invention, there is provided the solid pharmaceutical composition for oral administration prepared by a process described herein wherein the at least one additional pharmaceutically acceptable excipient comprises microcrystalline cellulose.

In illustrative embodiments of the present invention, there is provided the solid pharmaceutical composition for oral administration prepared by a process described herein wherein the at least one additional pharmaceutically acceptable excipient comprises magnesium stearate.

In illustrative embodiments of the present invention, there is provided the solid pharmaceutical composition for oral administration prepared by a process described herein wherein the at least one additional pharmaceutically acceptable excipient comprises colloidal silicon dioxide.

In illustrative embodiments of the present invention, there is provided the solid pharmaceutical composition for oral administration prepared by a process described herein wherein the aqueous environment is gastrointestinal fluid.

In illustrative embodiments of the present invention, there is provided the solid pharmaceutical composition for oral administration prepared by a process described herein wherein the mixing comprises mixing in a dry state.

In illustrative embodiments of the present invention, there is provided the solid pharmaceutical composition for oral administration prepared by a process described herein wherein the mixing is a wet granulation process.

In illustrative embodiments of the present invention, there is provided the solid pharmaceutical composition for oral administration prepared by a process described herein wherein the choline fenofibrate and the acid are mixed with water or a volatile organic solvent.

In illustrative embodiments of the present invention, there is provided the solid pharmaceutical composition for oral administration prepared by a process described herein wherein the volatile organic solvent is a lower alcohol.

In illustrative embodiments of the present invention, there is provided the solid pharmaceutical composition for oral administration prepared by a process described herein wherein the lower alcohol is methanol.

In illustrative embodiments of the present invention, there is provided the solid pharmaceutical composition for oral administration prepared by a process described herein further comprising drying after mixing.

In illustrative embodiments of the present invention, there is provided the solid pharmaceutical composition for oral administration prepared by a process described herein wherein the preparing comprises processing into granules or tablets.

Other aspects and features of the present invention will become apparent to those ordinarily skilled in the art upon review of the following description of specific embodiments of the invention in conjunction with the accompanying figures.

### DETAILED DESCRIPTION

Illustrative embodiments of the present invention include a solid pharmaceutical composition for oral administration prepared by a process comprising:

(a) mixing choline fenofibrate with an amount of an acid sufficient to substantially reduce a rate of dissolution of choline fenofibrate in gastrointestinal fluid at pH 4.5; and

(b) preparing a dosage form suitable for oral administration.

The acid may be one that has a solubility in water of less than 2 parts per 50 parts of water at 20 degrees Celsius. A non-limiting example of such an acid is fumaric acid.

As used herein, a choline fenofibrate dissolution rate or dissolution characteristic refers to a characteristic rate of dissolution of choline fenofibrate from a pharmaceutical composition according to the present invention, when tested in USP apparatus 2, at 100 rpm at 37 degrees Celsius. In some cases the test will be in 900 mL of 0.05M phosphate buffer, pH 4.5 and in other cases, the test will be in 900 mL of 0.05M phosphate buffer, pH 6.8; the context of each occurrence of the phrase makes it clear which pH the test is buffered to.

As used herein the word “about” indicates that strict adherence to the exact value to which the word about refers is not essential or required and that some minor variation is acceptable. In some occurrences, the word about may be substantially equivalent to +/−5%.

Illustrative embodiments of the present invention include a solid pharmaceutical composition for oral administration prepared by a process comprising:

(a) mixing choline fenofibrate and additional pharmaceutically acceptable excipients with an amount of an acid sufficient to substantially reduce a rate of dissolution of choline fenofibrate in gastrointestinal fluid at pH 4.5; and

(b) preparing a dosage form suitable for oral administration.

Additional pharmaceutically acceptable excipients may include, for example, binders (such as, for example, microcrystalline cellulose), lubricants (such as, for example, magnesium stearate), and flow agents (such as, for example, colloidal silicon dioxide).

Processes of the present invention may comprise mixing ingredients only in the dry state before further processing into granules or tablets. Alternatively, the process may comprise mixing the choline fenofibrate and acid, and optionally other excipients, in a wet granulation process, wherein the choline fenofibrate and acid are mixed with water or a volatile organic solvent and a mixture comprising the choline fenofibrate, the acid and water or volatile organic solvent is then dried and further processed. The volatile organic solvent may be a lower alcohol such as, for example, methanol.

Illustrative examples of the present invention include compositions that exhibit dissolution characteristics as follows, when tested in USP apparatus 2, at 100 rpm at 37 degrees Celsius:

(a) not more than about 40% at 2 hours in 900 mL of 0.05M phosphate buffer, pH 4.5; and

(b) not less than about 50% at 2 hours in 900 mL of 0.05M phosphate buffer pH 6.8

### EXAMPLES

The following examples are illustrative of some of the embodiments of the invention described herein. These examples should not be considered to limit the spirit or scope of the invention in any way.

### Example 1

Ingredients as follows were loaded into a high-shear granulator:

| | |
|---|---|
| Choline fenofibrate | 500.0 g |
| Fumaric acid | 137.6 g |
| Microcrystalline cellulose | 49.3 g |
| Total | 686.9 g |

125 g of methanol was added while blending, and blending was continued until the product temperature reached 80 degrees Celsius, thus producing dry granules.

### Example 2

Ingredients were mixed in the dry state as follows:

| | |
|---|---|
| Granules from example 1 | 136.4 g |
| Microcrystalline cellulose | 28.2 g |
| Magnesium stearate | 5.0 g |
| Colloidal silicon dioxide | 0.5 g |
| Total | 170.1 g |

The mixture was compressed into tablets of weight 34 mg each.

The amount of active ingredient in each tablet was thus 15 mg, expressed as fenofibric acid equivalent.

### Example 3

Capsules of 45 mg strength (expressed as fenofibric acid) were made by filling capsules with 3 tablets per capsule, using the tablets of example 2.

The capsules were tested for dissolution in USP apparatus 2 at 100 rpm at 37 degrees Celsius, with results as follows:

(a) about 10% at 2 hours in 900 mL of 0.05M phosphate buffer, pH 4.5; and

(b) about 70% at 2 hours in 900 mL of 0.05M phosphate buffer pH 6.8.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A solid pharmaceutical composition for oral administration comprising choline fenofibrate and an amount of an acid sufficient to substantially reduce a rate of dissolution of choline fenofibrate in an aqueous environment at pH 4.5 to less than about 50% in 2 hours.

2. The solid pharmaceutical composition of claim 1 wherein the acid has a solubility in water of less than 2 parts per 50 parts of water at 20 degrees Celsius.

3. The solid pharmaceutical composition of claim 1 wherein the acid is fumaric acid.

4. The solid pharmaceutical composition for oral administration of claim 1 wherein the aqueous environment is gastrointestinal fluid.

5. The solid pharmaceutical composition of claim 1 further comprising at least one additional pharmaceutically acceptable excipient.

6. The solid pharmaceutical composition of claim 5 wherein the at least one additional pharmaceutically acceptable excipient is selected from at least one of the group consisting of: binders, lubricants, flow agents and mixtures thereof.

7. The solid pharmaceutical composition of claim 5 wherein the at least one additional pharmaceutically acceptable excipient comprises microcrystalline cellulose.

8. The solid pharmaceutical composition of claim 5 wherein the at least one additional pharmaceutically acceptable excipient comprises magnesium stearate.

9. The solid pharmaceutical composition of claim 5 wherein the at least one additional pharmaceutically acceptable excipient comprises colloidal silicon dioxide.

10. The solid pharmaceutical composition of claim 1 wherein the pharmaceutical composition has a choline fenofibrate dissolution characteristic, when tested in USP apparatus 2, at 100 rpm at 37 degrees Celsius of not more than about 40% at 2 hours in 900 mL of 0.05M phosphate buffer, pH 4.5.

11. The solid pharmaceutical composition of claim 1 wherein the pharmaceutical composition has a choline fenofibrate dissolution characteristic, when tested in USP apparatus 2, at 100 rpm at 37 degrees Celsius of not less than about 50% at 2 hours in 900 mL of 0.05M phosphate buffer pH 6.8.

12. A solid pharmaceutical composition for oral administration prepared by a process comprising:
(a) mixing choline fenofibrate with an amount of an acid sufficient to substantially reduce a rate of dissolution of choline fenofibrate in an aqueous environment at pH 4.5 to less than about 50% in 2 hours; and
(b) preparing a dosage form suitable for oral administration.

13. The solid pharmaceutical composition for oral administration prepared by the process of claim 12 wherein the acid has a solubility in water of less than 2 parts per 50 parts of water at 20 degrees Celsius.

14. The solid pharmaceutical composition for oral administration prepared by the process of claim 12 wherein the acid is fumaric acid.

15. The solid pharmaceutical composition for oral administration prepared by the process of claim 12 further comprising mixing additional pharmaceutically acceptable excipients prior to preparing the dosage form suitable for oral administration.

16. The solid pharmaceutical composition for oral administration prepared by the process of claim 15 wherein the at least one additional pharmaceutically acceptable excipient is selected from at least one of the group consisting of: binders, lubricants, flow agents and mixtures thereof.

17. The solid pharmaceutical composition for oral administration prepared by the process of claim 15 wherein the at least one additional pharmaceutically acceptable excipient comprises microcrystalline cellulose.

18. The solid pharmaceutical composition for oral administration prepared by the process of claim 15 wherein the at least one additional pharmaceutically acceptable excipient comprises magnesium stearate.

19. The solid pharmaceutical composition for oral administration prepared by the process of claim 15 wherein the at least one additional pharmaceutically acceptable excipient comprises colloidal silicon dioxide.

20. The solid pharmaceutical composition for oral administration prepared by the process of claim 12 wherein the aqueous environment is gastrointestinal fluid.

21. The solid pharmaceutical composition for oral administration prepared by the process of claim 12 wherein the mixing comprises mixing in a dry state.

22. The solid pharmaceutical composition for oral administration prepared by the process of claim 12 wherein the mixing is a wet granulation process.

23. The solid pharmaceutical composition for oral administration prepared by the process of claim 22 wherein the choline fenofibrate and the acid are mixed with water or a volatile organic solvent.

24. The solid pharmaceutical composition for oral administration prepared by the process of claim 23 wherein the volatile organic solvent is a lower alcohol.

25. The solid pharmaceutical composition for oral administration prepared by the process of claim 24 wherein the lower alcohol is methanol.

26. The solid pharmaceutical composition for oral administration prepared by the process of claim 12 further comprising drying after mixing.

27. The solid pharmaceutical composition for oral administration prepared by the process of claim 12 wherein the preparing comprises processing into granules or tablets.

28. A solid pharmaceutical composition for oral administration comprising choline fenofibrate, an amount of fumaric acid sufficient to substantially reduce a rate of dissolution of choline fenofibrate in gastrointestinal fluid at pH 4.5 to less than about 50% in 2 hours, at least one pharmaceutically acceptable excipient selected from the group consisting of: microcrystalline cellulose, magnesium stearate, and colloidal silicon dioxide.
